(19) United States
(12) Patent Application Publication
(10) Pub. No.: US 2020/0316119 A1
(43) Pub. Date: Oct. 8, 2020

(54) CMV EPITOPES

(71) Applicant: The Counsil of the Queensland Institute of Medical REsearch, Herston (AU)

(72) Inventors: Rajiv KHANNA, Herston (AU); Corey Smith, Ashgrove (AU)

(21) Appl. No.: 16/303,677

(22) PCT Filed: May 23, 2017
(86) PCT No.: PCT/IB2017/000849

§ 371 Date: Nov. 21, 2018

Related U.S. Application Data
(60) Provisional application No. 62/340,223, filed on May 23, 2016.

(51) Int. Cl. A61K 35/17; C12N 15/86; A61K 39/12; A61P 31/20; A61P 35/00

(52) U.S. Cl. CPC A61K 35/17; C12N 15/86; A61K 2039/572; A61P 31/20; A61P 35/00; A61K 39/12

(57) ABSTRACT

Provided herein are compositions and methods related to the treatment of a CMV infection and/or cancer in a subject.

### RELATED APPLICATIONS

This application claims the benefit of priority to U.S. Provisional Patent Application Ser. No. 62/340,223, filed May 23, 2016, hereby incorporated by reference in its entirety.

### BACKGROUND

Cytomegalovirus (CMV, also known as human herpesvirus-5) is a nearly ubiquitous herpes virus that infects between 60% and 90% of individuals. Following primary infection, CMV typically establishes a persistent infection that is kept under control by a healthy immune system. CMV employs a multitude of immune-modulatory strategies to evade the host immune response. Examples of such strategies include inhibition of interferon (IFN) and IFN-stimulated genes, degradation of HLA to prevent antigen presentation to cytotoxic T cells and modulation of activating and inhibitory ligands to prevent natural killer (NK) cell function.

Though CMV infection typically goes unnoticed in healthy individuals, reactivation from viral latency in immunocompromised individuals (e.g., HIV-infected persons, organ transplant recipients), or acquisition of primary infection in such individuals (e.g., during transplantation) can lead to serious disease. For example, CMV is one of the major causes of graft failure and mortality in transplant recipients who require prolonged immunosuppression, and CMV infection during pregnancy can lead to congenital abnormalities. CMV infection has also been linked with cancer, even in immunocompetent individuals.

CMV infection in immunocompromised individuals is currently treated using purified plasma immunoglobulin (CMV-IGIV) and antiviral drugs, such as ganciclovir (Cytovene) and valganciclovir (Valcyte). Because CMV-IVIG is derived from donated human plasma, it is difficult to produce in large quantities and its use carries the risk of the transmission of infectious disease. Drug-resistant CMV strains have become increasingly common, often rendering current therapies ineffective. Recent attempts to develop a CMV vaccine have proven unsuccessful. Thus, there is a great need for new and improved methods and compositions for the treatment of CMV and CMV-associated cancers.

### SUMMARY

Provided herein are compositions and methods related to CMV epitopes (e.g., CMV epitopes listed in Table 1) that are recognized by cytotoxic T lymphocytes (CTLs) and that are useful in the prevention and/or treatment of CMV infection and/or cancer (e.g., a cancer expressing a CMV epitope provided herein).

In certain aspects, provided herein are compositions (e.g., therapeutic compositions, such as vaccine compositions) containing a polypeptide comprising one or more of the CMV epitopes described herein (e.g., CMV epitopes listed in Table 1) and/or a nucleic acid encoding such a polypeptide, as well as methods of treating and/or preventing CMV infection and/or cancer by administering such compositions to a subject. In some embodiments, the polypeptide is not a full-length CMV protein. In some embodiments, the polypeptide contains no more than 15, 20, 25, 30, 35 or 40 contiguous amino acid of a full-length CMV protein. In some embodiments, the polypeptide consists essentially of a CMV epitope described herein. In some embodiments, the polypeptide consists of a CMV epitope described herein. In some embodiments, the polypeptide is no more than 15, 20, 25, 30, 35 or 40 amino acids in length. In some embodiments, the composition further comprises an adjuvant.

In some aspects, provided herein are methods of generating, activating and/or inducing proliferation of CTLs that recognize one or more of the CMV epitopes described herein, for example, by incubating a sample comprising CTLs (i.e., a PBMC sample) with antigen-presenting cells (APCs) that present one or more of the CMV epitopes described herein (e.g., APCs that present a peptide comprising a CMV epitope described herein on a class I MHC complex). In some embodiments, the APCs are autologous to the subject from whom the CTLs were obtained. In some embodiments, the APCs are not autologous to the subject from whom the CTLs were obtained. In some embodiments the APCs are B cells, antigen-presenting T-cells, dendritic cells, or artificial antigen-presenting cells (e.g., aK562 cells). In some aspects, the antigen-presenting cells (e.g., aK562 cells) express CD80, CD83, 41BB-L, and/or CD86.

In some aspects, provided herein are compositions (e.g., therapeutic compositions) comprising CTLs that recognize one or more of the CMV epitopes described herein (i.e., CTLs expressing a T cell receptor (TCR) that binds to a peptide comprising a CMV epitope described herein that is presented on a class I MHC complex), as well as methods of treating and/or preventing CMV infection and/or cancer by administering such compositions to a subject. For example, in some embodiments, provided herein is a method for treating and/or preventing a cancer and/or a CMV infection in a subject, comprising administering to the subject a composition comprising CTLs that recognize one or more of the CMV epitopes described herein. In some embodiments, the CTLs are not autologous to the subject. In some embodiments, the T cells are autologous to the subject. In some embodiments, the CTLs are stored in a cell bank before they are administered to the subject. In some embodiments, the method further comprises generating, activating and/or inducing proliferation of the CTLs using a method described herein. In some aspects, provided herein is a T cell (e.g., a CTL) expressing a T cell receptor (TCR) that binds to a peptide listed in Table 1 presented on a major histocompatibility complex (MHC).

In some embodiments, provided herein are APCs that present one or more peptides comprising a CMV epitope described herein (e.g., APCs that present one or more of the CMV epitopes on a class I MHC). In certain aspects, provided herein are methods of generating APCs that present the one or more of the CMV epitopes described herein comprising contacting an APC with a peptide comprising a CMV epitope described herein and/or with a nucleic acid encoding a CMV epitope described herein. In some embodiments, the APCs are not autologous to the subject from whom the CTLs were obtained. In some embodiments the APCs are B cells, antigen-presenting T-cells, dendritic cells, or artificial antigen-presenting cells (e.g., aK562 cells). In some aspects, the antigen presenting cells (e.g., aK562 cells) express CD80, CD83, 41BB-L, and/or CD86. In some embodiments, provided herein are methods of treating or preventing cancer and/or a CMV infection in a subject comprising the step of administering to a subject the APCs described herein.

In certain aspects, provided herein are antigen-binding molecules (e.g., antibodies, antibody fragments, TCRs, chimeric antigen receptors (CARs)) that specifically bind to a CMV epitope described herein. In some embodiments, the antigen-binding molecule is an antibody or an antigen-binding fragment thereof. In some embodiments, the antibody is a chimeric antibody, a humanized antibody or a fully human antibody. In some embodiments, the antibody or antigen-binding fragment thereof is a full length immunoglobulin molecule, an scFv, a Fab fragment, an Fab′ fragment, a F(ab′)2 fragment, an Fv, a camelid or a disulfide linked Fv. In some embodiments, the antibody binds to the epitope provided herein with a dissociation constant of no greater than about 10⁻⁷ M, 10⁻⁸ M or 10⁻⁹M. In some embodiments, the antigen-binding molecule is conjugated to a drug (e.g., as part of an antibody-drug conjugate). In some embodiments, the antigen-binding molecule is linked to a cytotoxic agent (e.g., MMAE, DM-1, a maytansinoid, a doxorubicin derivative, an auristatin, a calcheamicin, CC-1065, aduocarmycin or a anthracycline). In some embodiments, the antigen-binding molecule is linked to an antiviral agent (e.g., ganciclovir, valganciclovir, foscarnet, cidofovir, acyclovir, formivirsen, maribavir, BAY 38-4766 or GW275175X). In some embodiments, provided herein are methods of treating cancer and/or a CMV infection in a subject comprising administering to the subject an antigen-binding molecule disclosed herein.

In some aspects, provided herein are nucleic acids comprising a sequence encoding one or more of the peptides provided herein. In some embodiments, the sequence encoding one or more of the peptides provided herein is operably linked to one or more regulatory sequences In some embodiments, the nucleic acid is an expression vector. In some embodiments, the nucleic acid is an adenoviral vector.

In some aspects, provided herein are pharmaceutical compositions comprising the CMV peptides, CTLs, APCs, nucleic acids, and/or antigen-binding molecules described herein and a pharmaceutical acceptable carrier. In some embodiments, provided herein are methods for treating and/or preventing CMV infection and/or cancer in a subject by administering a pharmaceutical composition provided herein.

In some aspects, provided herein is a method of identifying a subject suitable for a method of treatment provided herein (e.g., administration of CTLs, APCs, polypeptides, compositions, antibodies or nucleic acids described herein) comprising isolating a sample from the subject and detecting the presence of a CMV epitope provided herein or a nucleic acid encoding a CMV epitope provide herein the sample (e.g., a blood or tumor sample). In some embodiments, the CMV epitope provided herein is detected by contacting the sample with an antigen-binding molecule provided herein. In some embodiments, the subject identified as being suitable for a method of treatment provided herein is treated using the method of treatment.

### BRIEF DESCRIPTION OF THE DRAWINGS

FIG. 1 shows pyrosequencing analysis of the IE-1 sequence variants in hematopoietic stem cell transplant (HSCT) recipients.

FIG. 2 shows the kinetics of variant-specific T cell activation following viral reactivation in HSCT transplant recipients.

FIG. 3 shows functional avidity analysis of IE-1 variant specific T cell populations.

FIG. 4 shows the effect of co-infection on viral reactivations and the association of viral reactivation with overall T cell immunity.

### DETAILED DESCRIPTION

### General

Provided herein are compositions and methods related to CMV epitopes (e.g., CMV epitopes listed in Table 1) that are recognized by cytotoxic T lymphocytes (CTLs) and that are useful in the prevention and/or treatment of CMV infection and/or cancer. In certain aspects, provided herein are compositions (e.g., therapeutic compositions, such as vaccine compositions) containing a polypeptide comprising one or more of the CMV epitopes described herein (e.g., CMV epitopes listed in Table 1), nucleic acids encoding such a polypeptide, CTLs that recognize such a peptide, APCs presenting such peptides and/or antigen-binding molecules that bind specifically to such peptides, as well as methods of treating and/or preventing CMV infection and/or cancer by administering such compositions to a subject. In some embodiments, also provided herein are methods of identifying a subject suitable for treatment according to a method provided herein.

### Definitions

For convenience, certain terms employed in the specification, examples, and appended claims are collected here.

The articles “a” and “an” are used herein to refer to one or to more than one (i.e., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering. Such an agent can contain, for example, peptide described herein, an antigen presenting cell provided herein and/or a CTL provided herein.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments.

The terms “antigen-binding fragment” and “antigen-binding portion” of an antibody, as used herein, refers to one or more fragments of an antibody that retain the ability to bind to an antigen. Examples of binding fragments encompassed within the term “antigen-binding fragment” of an antibody include Fab, Fab′, F(ab′)₂, Fv, scFv, disulfide linked Fv, Fd, diabodies, single-chain antibodies, camelid antibodies, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

The term “binding” or “interacting” refers to an association, which may be a stable association, between two molecules, e.g., between a peptide and a binding partner or agent, e.g., small molecule, due to, for example, electrostatic, hydrophobic, ionic and/or hydrogen-bond interactions under physiological conditions.

The term “biological sample,” “tissue sample,” or simply “sample” each refers to a collection of cells obtained from a tissue of a subject. The source of the tissue sample may be solid tissue, as from a fresh, frozen and/or preserved organ, tissue sample, biopsy, or aspirate; blood or any blood constituents, serum, blood; bodily fluids such as cerebral spinal fluid, amniotic fluid, peritoneal fluid or interstitial fluid, urine, saliva, stool, tears; or cells from any time in gestation or development of the subject.

As used herein, the term “cancer” includes, but is not limited to, solid tumors and blood borne tumors. The term cancer includes diseases of the skin, tissues, organs, bone, cartilage, blood and vessels. The term “cancer” further encompasses primary and metastatic cancers.

The term “epitope” means a protein determinant capable of specific binding to an antibody. Epitopes usually consist of chemically active surface groupings of molecules such as amino acids or sugar side chains. Certain epitopes can be defined by a particular sequence of amino acids to which a T cell receptor or antibody is capable of binding.

The term “isolated nucleic acid” refers to a polynucleotide of natural or synthetic origin or some combination thereof, which (1) is not associated with the cell in which the “isolated nucleic acid” is found in nature, and/or (2) is operably linked to a polynucleotide to which it is not linked in nature.

The term “isolated polypeptide” refers to a polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides.

As used herein, a therapeutic that “prevents” a condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

As used herein, “specific binding” refers to the ability of an antibody to bind to a predetermined antigen or the ability of a peptide to bind to its predetermined binding partner. Typically, an antibody or peptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, casein).

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment.

“Treating” a disease in a subject or “treating” a subject having a disease refers to subjecting the subject to a pharmaceutical treatment, e.g., the administration of a drug, such that at least one symptom of the disease is decreased or prevented from worsening.

The term “vector” refers to the means by which a nucleic acid can be propagated and/or transferred between organisms, cells, or cellular components. Vectors include plasmids, viruses, bacteriophage, pro-viruses, phagemids, transposons, and artificial chromosomes, and the like, that may or may not be able to replicate autonomously or integrate into a chromosome of a host cell.

### Peptides

Provided herein are peptides comprising CMV epitopes that are recognized by cytotoxic T lymphocytes (CTLs) and that are useful in the prevention and/or treatment of CMV infection and/or cancer (e.g., a cancer expressing a CMV epitope provided herein). In certain embodiments, the CMV epitope is an epitope listed in Table 1.

TABLE 1
Exemplary CMV epitopes

| Epitope | SEQ ID NO.: |
|---|---|
| KARAKKDELR | |
| ARAKKDELR | |
| RRKMMYMYCR | |
| KARAKKDELK | |
| ARAKKDELK | |
| KRKMIYMYCR | |
| VLEETSVML | |
| YILEETSVML | |
| DELRRKMMY | |
| DELKRKMIY | |
| EEAIAVAYL | |
| EDAIAAYTL | |
| ELRRKMMYM | |
| ELKRKMIYM | |
| AYAQKIFKIL | |
| TYSQKIFKIL | |
| KARAKKDELR | |
| KARAKKDELK | |
| ARAKKDELK | |
| ARAKKDELR | |
| KRKMIYMCYR | |
| RRKMMYMCYR | |
| FMDILTTCV | |
| NLVPMVATV | |
| RPHERNGFTVL | |
| TPRVTGGGAM | |
| VTEHDTLLY | |
| QIKVRVDMV | |
| YSEHPTFTSQY | |

In some embodiments, the peptides provided herein are full length CMV proteins. In some embodiments, the peptides provided herein comprise less than 100, 90, 80, 70, 60, 50, 40, 30, 25, 20, 15 or 10 contiguous amino acids of the CMV viral protein. In some embodiments, the peptides provided herein comprise two or more of the CMV epitopes listed in Table 1. For example, in some embodiments, the peptide provided herein comprises two or more of the CMV epitopes listed in table 1 connected by polypeptide linkers. In some embodiments, the peptide provided herein comprises 2, 3, 4, 5, 6, 7, 8, 9, 10, 11, 12, 13, 14, 15, 16, 17, 18, 19 or 20 of the epitopes listed in Table 1.

In some embodiments, the peptide provided herein consists of an epitope listed in Table 1. In some embodiments, the peptide provided herein consists essentially of an epitope listed in Table 1. In some embodiments, the peptide provided herein comprise no more than 20, 19, 18, 17, 16, 15, 14, 13, 12, 11, 10, 9, 8, 7, 6, 5, 4, 3, 2 or 1 amino acids in addition to the epitopes listed in Table 1.

In some embodiments, the sequence of the peptides comprise an EBV viral protein sequence except for 1 or more (e.g., 1, 2, 3, 4, 5, 6, 7, 8, 9, 10 or more) conservative sequence modifications. As used herein, the term “conservative sequence modifications” is intended to refer to amino acid modifications that do not significantly affect or alter the interaction between a TCR and a peptide containing the amino acid sequence presented on an MHC. Such conservative modifications include amino acid substitutions, additions (e.g., additions of amino acids to the N or C terminus of the peptide) and deletions (e.g., deletions of amino acids from the N or C terminus of the peptide). Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues of the peptides described herein can be replaced with other amino acid residues from the same side chain family and the altered peptide can be tested for retention of TCR binding using methods known in the art. Modifications can be introduced into an antibody by standard techniques known in the art, such as site-directed mutagenesis and PCR-mediated mutagenesis.

To determine the percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-identical sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

Also provided herein are chimeric or fusion proteins. As used herein, a “chimeric protein” or “fusion protein” comprises a peptide(s) provided herein (e.g., those comprising an epitope listed in Table 1) linked to a distinct peptide to which it is not linked in nature. For example, the distinct peptide can be fused to the N-terminus or C-terminus of the peptide either directly, through a peptide bond, or indirectly through a chemical linker. In some embodiments, the peptide of the provided herein is linked to polypeptides comprising other CMV epitopes. In some embodiments, the peptide provided herein is linked to peptides comprising epitopes from other viral and/or infectious diseases. In some embodiments, the peptide provided herein is linked to a peptide encoding a cancer-associated epitope.

A chimeric or fusion peptide provided herein can be produced by standard recombinant DNA techniques. For example, DNA fragments coding for the different peptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see, for example, Current Protocols in Molecular Biology, Ausubel et al., eds., John Wiley & Sons: 1992). Moreover, many expression vectors are commercially available that already encode a fusion moiety.

In some aspects, provided herein are cells that present a peptide described herein (e.g., a peptide comprising an epitope listed in Table 1). In some embodiments, the cell is a mammalian cell. In some embodiments the cell is an antigen presenting cell (APC) (e.g., an antigen presenting t-cell, a dendritic cell, a B cell, a macrophage or am artificial antigen presenting cell, such as aK562 cell). A cell presenting a peptide described herein can be produced by standard techniques known in the art. For example, a cell may be pulsed to encourage peptide uptake. In some embodiments, the cells are transfected with a nucleic acid encoding a peptide provided herein. In some aspects, provided herein are methods of producing antigen presenting cells (APCs), comprising pulsing a cell with the peptides described herein. Exemplary examples of producing antigen presenting cells can be found in WO2013088114, hereby incorporated in its entirety.

The peptides provided herein can be isolated from cells or tissue sources by an appropriate purification scheme using standard protein purification techniques, can be produced by recombinant DNA techniques, and/or can be chemically synthesized using standard peptide synthesis techniques. The peptides described herein can be produced in prokaryotic or eukaryotic host cells by expression of nucleotides encoding a peptide(s) of the present invention. Alternatively, such peptides can be synthesized by chemical methods. Methods for expression of heterologous peptides in recombinant hosts, chemical synthesis of peptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11:255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference.

### Nucleic Acid Molecules

Provided herein are nucleic acid molecules that encode the peptides described herein. In some aspects, provided herein are methods of treating cancer or CMV by administering to a subject the nucleic acids disclosed herein. The nucleic acids may be present, for example, in whole cells, in a cell lysate, or in a partially purified or substantially pure form.

In some embodiments, provided herein are vectors (e.g., a viral vector, such as an adenovirus based expression vector) that contain the nucleic acid molecules described herein. As used herein, the term “vector,” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication, episomal mammalian vectors). Other vectors (e.g., non-episomal mammalian vectors) can be integrated into the genome of a host cell upon introduction into the host cell, and thereby be replicated along with the host genome. Moreover, certain vectors are capable of directing the expression of genes. Such vectors are referred to herein as “recombinant expression vectors” (or simply, “expression vectors”). In some embodiments, provided herein are nucleic acids operable linked to one or more regulatory sequences (e.g., a promoter) in an expression vector. In some embodiments the cell transcribes the nucleic acid provided herein and thereby expresses an antibody, antigen binding fragment thereof or peptide described herein. The nucleic acid molecule can be integrated into the genome of the cell or it can be extrachromosomal.

In some embodiments, the nucleic acid provided herein is part of a vaccine. In some embodiments, the vaccine is delivered to a subject in a vector, including, but not limited to, a bacterial vector and/or a viral vector. Examples of bacterial vectors include, but are not limited to, Mycobacterium bovis (BCG), Salmonella Typhimurium ssp., Salmonella Typhi ssp., Clostridium sp. spores, Escherichia coli Nissle 1917, Escherichia coli K-12/LLO, Listeria monocytogenes, and Shigella flexneri. Examples of viral vectors include, but are not limited to, vaccinia, adenovirus, RNA viruses (replicons), and replication-defective like avipox, fowlpox, canarypox, MVA, and adenovirus.

In some embodiments, provided herein are cells that contain a nucleic acid described herein (e.g., a nucleic acid encoding an antibody, antigen binding fragment thereof or peptide described herein). The cell can be, for example, prokaryotic, eukaryotic, mammalian, avian, murine and/or human. In some embodiments, the cell is a mammalian cell. In some embodiments the cell is an APC (e.g. an antigen presenting T cell, a dendritic cell, a B cell, or an aK562 cell). In the present methods, a nucleic acid described herein can be administered to the cell, for example, as nucleic acid without delivery vehicle, in combination with a delivery reagent. In some embodiments, any nucleic acid delivery method known in the art can be used in the methods described herein. Suitable delivery reagents include, but are not limited to, e.g., the Mirus Transit TKO lipophilic reagent; lipofectin; lipofectamine; cellfectin; polycations (e.g., polylysine), atelocollagen, nanoplexes and liposomes. In some embodiments of the methods described herein, liposomes are used to deliver a nucleic acid to a cell or subject. Liposomes suitable for use in the methods described herein can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

### Antibodies

In some aspects, the compositions and methods provided herein relate to antibodies and antigen-binding fragments thereof that bind specifically to a protein expressed on the plasma membrane of a CMV infected cell or a cancer cell (e.g., a protein comprising the epitope listed in Table 1). In some embodiments, the antibodies bind to a particular epitope of one of the peptides provided herein. In some embodiments, an antibody that binds to a CMV protein comprising an epitope with an amino acid sequence in Table 1, wherein the CMV protein is not a full length CMV protein. In some embodiments, the epitope is an extracellular epitope. In some embodiments, the epitope is an epitope listed in Table 1. In some embodiments, the antibodies can be polyclonal or monoclonal and can be, for example, murine, chimeric, humanized or fully human. In some embodiments, the antibody is a full length immunoglobulin molecule, an scFv, a Fab fragment, an Fab′ fragment, a F(ab′)2 fragment, an Fv, a camelid antibody or a disulfide linked Fv.

Polyclonal antibodies can be prepared by immunizing a suitable subject (e.g. a mouse) with a peptide immunogen (e.g., an amino acid sequence listed in Table 1). In some embodiments, the peptide immunogen comprises an extracellular epitope of a target protein provided herein. The peptide antibody titer in the immunized subject can be monitored over time by standard techniques, such as with an enzyme linked immunosorbent assay (ELISA) using immobilized peptide. If desired, the antibody directed against the antigen can be isolated from the mammal (e.g., from the blood) and further purified by well known techniques, such as protein A chromatography to obtain the IgG fraction.

At an appropriate time after immunization, e.g., when the antibody titers are highest, antibody-producing cells can be obtained from the subject and used to prepare monoclonal antibodies using standard techniques, such as the hybridoma technique originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; and Yeh et al. (1982) Int. J. Cancer 29:269-75), a human B cell hybridoma technique (Kozbor et al. (1983) Immunol. Today 4:72), an EBV-hybridoma technique (Cole et al. (1985) Monoclonal Antibodies and Cancer Therapy, Alan R. Liss, Inc., pp. 77-96) or a trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the peptide antigen, preferably specifically.

As an alternative to preparing monoclonal antibody-secreting hybridomas, a monoclonal antibody that binds to a target protein described herein can be obtained by screening a recombinant combinatorial immunoglobulin library with the appropriate peptide (e.g. a peptide comprising an epitope of Table 1) to thereby isolate immunoglobulin library members that bind the peptide.

Additionally, recombinant antibodies specific for a target protein provided herein and/or an extracellular epitope of a target protein provided herein, such as chimeric or humanized monoclonal antibodies, can be made using standard recombinant DNA techniques. Such chimeric and humanized monoclonal antibodies can be produced by recombinant DNA techniques known in the art, for example using methods described in U.S. Pat. Nos. 4,816,567; 5,565,332; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Human monoclonal antibodies specific for a target protein provided herein and/or an extracellular epitope provided herein can be generated using transgenic or transchromosomal mice carrying parts of the human immune system rather than the mouse system. For example, “HuMAb mice” which contain a human immunoglobulin gene miniloci that encodes unrearranged human heavy (μ and γ) and κ light chain immunoglobulin sequences, together with targeted mutations that inactivate the endogenous μ and κ chain loci (Lonberg, N. et al. (1994) Nature 368(6474): 856 859). Accordingly, the mice exhibit reduced expression of mouse IgM or κ, and in response to immunization, the introduced human heavy and light chain transgenes undergo class switching and somatic mutation to generate high affinity human IgGκ monoclonal antibodies (Lonberg, N. et al. (1994), supra; reviewed in Lonberg, N. (1994) Handbook of Experimental Pharmacology 113:49 101; Lonberg, N. and Huszar, D. (1995) Intern. Rev. Immunol. Vol. 13: 65 93, and Harding, F. and Lonberg, N. (1995) Ann. N. Y Acad. Sci 764:536 546). The preparation of HuMAb mice is described in Taylor, L. et al. (1992) Nucleic Acids Research 20:6287 6295; Chen, J. et al. (1993) International Immunology 5: 647 656; Tuaillon et al. (1993) Proc. Natl. Acad. Sci USA 90:3720 3724; Choi et al. (1993) Nature Genetics 4:117 123; Chen, J. et al. (1993) EMBO J. 12: 821 830; Tuaillon et al. (1994) J. Immunol. 152:2912 2920; Lonberg et al., (1994) Nature 368(6474): 856 859; Lonberg, N. (1994) Handbook of Experimental Pharmacology 113:49 101; Taylor, L. et al. (1994) International Immunology 6: 579 591; Lonberg, N. and Huszar, D. (1995) Intern. Rev. Immunol. Vol. 13: 65 93; Harding, F. and Lonberg, N. (1995) Ann. N.Y. Acad. Sci 764:536 546; Fishwild, D. et al. (1996) Nature Biotechnology 14: 845 851. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807.

In some embodiments, the antibodies provided herein are able to bind to an epitope listed in Tables 1 with a dissociation constant of no greater than 10⁻⁶, 10⁻⁷, 10⁻⁸ or 10⁻⁹ M. Standard assays to evaluate the binding ability of the antibodies are known in the art, including for example, ELISAs, Western blots and RIAs. The binding kinetics (e.g., binding affinity) of the antibodies also can be assessed by standard assays known in the art, such as by Biacore analysis.

In some embodiments the antibody is part of an antibody-drug conjugate. Antibody-drug conjugates are therapeutic molecules comprising an antibody (e.g., an antibody that binds to a protein listed in Table 1) linked to a biologically active agent, such as a cytotoxic agent or an antiviral agent. In some embodiments, the biologically active agent is linked to the antibody via a chemical linker. Such linkers can be based on any stable chemical motif, including disulfides, hydrazones, peptides or thioethers. In some embodiments, the linker is a cleavable linker and the biologically active agent is released from the antibody upon antibody binding to the plasma membrane target protein. In some embodiments, the linker is a noncleavable linker.

In some embodiments, the antibody-drug conjugate comprises an antibody linked to a cytotoxic agent. In some embodiments, any cytotoxic agent able to kill CMV infected cells can be used. In some embodiments, the cytotoxic agent is MMAE, DM-1, a maytansinoid, a doxorubicin derivative, an auristatin, a calcheamicin, CC-1065, an aduocarmycin or an anthracycline.

In some embodiments, the antibody-drug conjugate comprises an antibody linked to an antiviral agent. In some embodiments, any antiviral agent capable of inhibiting CMV replication is used. In some embodiments, the antiviral agent is ganciclovir, valganciclovir, foscarnet, cidofovir, acyclovir, formivirsen, maribavir, BAY 38-4766 or GW275175X. In some embodiments, provided herein are vaccines composing the antibodies or antibody-drug conjugates described herein.

### Cells

In some aspects, provided herein are antigen presenting cells (APCs) that express on their surface a MHC that present one or more peptides comprising a CMV epitope described herein (e.g., APCs that present one or more of the CMV epitopes listed in Table 1). In some embodiments, the MHC is a class I MHC. In some embodiments, the MHC is a class II MHC. In some embodiments, the class I MHC has an α chain polypeptide that is HLA-A, HLA-B, HLA-C, HLA-E, HLA-F, HLA-g, HLA-K or HLA-L. In some embodiment, the class II MHC has an α chain polypeptide that is HLA-DMA, HLA-DOA, HLA-DPA, HLA-DQA or HLA-DRA. In some embodiments, the class II MHC has a β chain polypeptide that is HLA-DMB, HLA-DOB, HLA-DPB, HLA-DQB or HLA-DRB.

In some embodiments the APCs are B cells, antigen presenting T-cells, dendritic cells, or artificial antigen-presenting cells (e.g., aK562 cells). Dendritic cells for use in the process may be prepared by taking PBMCs from a patient sample and adhering them to plastic. Generally the monocyte population sticks and all other cells can be washed off. The adherent population is then differentiated with IL-4 and GM-CSF to produce monocyte derived dendritic cells. These cells may be matured by the addition of IL-1β, IL-6, PGE-1 and TNF-α (which upregulates the important co-stimulatory molecules on the surface of the dendritic cell) and are then transduced with one or more of the peptides provided herein.

In some embodiments, the APC is an artificial antigen-presenting cell, such as an aK562 cell. In some embodiments, the artificial antigen-presenting cells are engineered to express CD80, CD83, 41BB-L, and/or CD86. Exemplary artificial antigen-presenting cells, including aK562 cells, are described U.S. Pat. Pub. No. 2003/0147869, which is hereby incorporated by reference.

In certain aspects, provided herein are methods of generating APCs that present the one or more of the CMV epitopes described herein comprising contacting an APC with a peptide comprising a CMV epitope described herein and/or with a nucleic acid encoding a CMV epitope described herein. In some embodiments, the APCs are irradiated.

In certain aspects, provided herein are T cells (e.g., CD4 T cells and/or CD8 T cells) that express a TCR (e.g., an αβ TCR or a γδ TCR) that recognizes a peptide described herein (a peptide comprising a CMV epitopes listed in Table 1) presented on a MHC. In some embodiments, the T cell is a CD8 T cell (a CTL) that expresses a TCR that recognizes a peptide described herein presented on a class I MHC. In some embodiments, the T cell is a CD4 T cell (a helper T cell) that recognizes a peptide described herein presented on a class II MHC.

In some aspects, provided herein are methods of generating, activating and/or inducing proliferation of T cells (e.g., CTLs) that recognize one or more of the CMV epitopes described herein. In some embodiments, a sample comprising CTLs (i.e., a PBMC sample) is incubated in culture with an APC provided herein (e.g., an APCs that present a peptide comprising a CMV epitope described herein on a class I MHC complex). In some embodiments, the APCs are autologous to the subject from whom the T cells were obtained. In some embodiments, the sample containing T cells are incubated 2 or more times with APCs provided herein. In some embodiments, the T cells are incubated with the APCs in the presence of at least one cytokine. In some embodiments, the cytokine is IL-4, IL-7 and/or IL-15. Exemplary methods for inducing proliferation of T cells using APCs are provided, for example, in U.S. Pat. Pub. No. 2015/0017723, which is hereby incorporated by reference.

In some aspects, provided herein are compositions (e.g., therapeutic compositions) comprising T cells and/or APCs provided herein. In some embodiments, such compositions are used to treat and/or prevent a cancer and/or a CMV infection in a subject by administering to the subject an effective amount of the composition In some embodiments, the T cells and/or APCs are not autologous to the subject. In some embodiments, the T cells and/or APCs are autologous to the subject. In some embodiments, the T cells and/or APCs are stored in a cell bank before they are administered to the subject.

### Pharmaceutical Compositions

In some aspects, provided herein is a composition (e.g., a pharmaceutical composition, such as a vaccine composition), containing a peptide (e.g., comprising an epitope from Table 1), nucleic acid, antibody, CTL, or an APC described herein formulated together with a pharmaceutically acceptable carrier, as well as methods of treating cancer or a CMV infection using such pharmaceutical compositions. In some embodiments, the composition includes a combination of multiple (e.g., two or more) agents provided herein.

In some embodiments, the pharmaceutical composition further comprises an adjuvant. As used herein, the term “adjuvant” broadly refers to an agent that affects an immunological or physiological response in a patient or subject. For example, an adjuvant might increase the presence of an antigen over time or to an area of interest like a tumor, help absorb an antigen-presenting cell antigen, activate macrophages and lymphocytes and support the production of cytokines. By changing an immune response, an adjuvant might permit a smaller dose of an immune interacting agent to increase the effectiveness or safety of a particular dose of the immune interacting agent. For example, an adjuvant might prevent T cell exhaustion and thus increase the effectiveness or safety of a particular immune interacting agent. Examples of adjuvants include, but are not limited to, an immune modulatory protein, Adjuvant 65, α-GalCer, aluminum phosphate, aluminum hydroxide, calcium phosphate, β-Glucan Peptide, CpG DNA, GPI-0100, lipid A, lipopolysaccharide, Lipovant, Montanide, N-acetyl-muramyl-L-alanyl-D-isoglutamine, Pam3CSK4, quil A and trehalose dimycolate.

Methods of preparing these formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions of this invention suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, the agents of the present invention, which may be used in a suitable hydrated form, and/or the pharmaceutical compositions of the present invention, are formulated into pharmaceutically-acceptable dosage forms by conventional methods known to those of skill in the art.

### Therapeutic Methods

In certain embodiments, provided herein are methods of treating a CMV infection and/or a cancer in a subject comprising administering to the subject a pharmaceutical composition provided herein.

In some embodiments, provided herein is a method of treating a CMV infection in a subject. In some embodiments, the subject treated is immunocompromised. For example, in some embodiments, the subject has a T cell deficiency. In some embodiments, the subject has leukemia, lymphoma or multiple myeloma. In some embodiments, the subject is infected with HIV and/or has AIDS. In some embodiments, the subject has undergone a tissue, organ and/or bone marrow transplant. In some embodiments, the subject is being administered immunosuppressive drugs. In some embodiments, the subject has undergone and/or is undergoing a chemotherapy. In some embodiments, the subject has undergone and/or is undergoing radiation therapy.

In some embodiments, the subject is also administered an anti-viral drug that inhibits CMV replication. For example, in some embodiments, the subject is administered ganciclovir, valganciclovir, foscarnet, cidofovir, acyclovir, formivirsen, maribavir, BAY 38-4766 or GW275175X.

In some embodiments, the subject has cancer. In some embodiments, the methods described herein may be used to treat any cancerous or pre-cancerous tumor. In some embodiments, the cancer expresses one or more of the CMV epitopes provided herein (e.g., the CMV epitopes listed in Table 1). In some embodiments, the cancer includes a solid tumor. Cancers that may be treated by methods and compositions provided herein include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometrioid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; mammary paget's disease; acinar cell carcinoma; adenosquamous carcinoma; adenocarcinoma w/squamous metaplasia; malignant thymoma; malignant ovarian stromal tumor; malignant thecoma; malignant granulosa cell tumor; and malignant roblastoma; sertoli cell carcinoma; malignant leydig cell tumor; malignant lipid cell tumor; malignant paraganglioma; malignant extra-mammary paraganglioma; pheochromocytoma; glomangiosarcoma; malignant melanoma; amelanotic melanoma; superficial spreading melanoma; malignant melanoma in giant pigmented nevus; epithelioid cell melanoma; malignant blue nevus; sarcoma; fibrosarcoma; malignant fibrous histiocytoma; myxosarcoma; liposarcoma; leiomyosarcoma; rhabdomyosarcoma; embryonal rhabdomyosarcoma; alveolar rhabdomyosarcoma; stromal sarcoma; malignant mixed tumor; mullerian mixed tumor; nephroblastoma; hepatoblastoma; carcinosarcoma; malignant mesenchymoma; malignant brenner tumor; malignant phyllodes tumor; synovial sarcoma; malignant mesothelioma; dysgerminoma; embryonal carcinoma; malignant teratoma; malignant struma ovarii; choriocarcinoma; malignant mesonephroma; hemangiosarcoma; malignant hemangioendothelioma; kaposi's sarcoma; malignant hemangiopericytoma; lymphangiosarcoma; osteosarcoma; juxtacortical osteosarcoma; chondrosarcoma; malignant chondroblastoma; mesenchymal chondrosarcoma; giant cell tumor of bone; ewing's sarcoma; malignant odontogenic tumor; ameloblastic odontosarcoma; malignant ameloblastoma; ameloblastic fibrosarcoma; malignant pinealoma; chordoma; malignant glioma; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; malignant meningioma; neurofibrosarcoma; malignant neurilemmoma; malignant granular cell tumor; malignant lymphoma; Hodgkin's disease; Hodgkin's lymphoma; paragranuloma; small lymphocytic malignant lymphoma; diffuse large cell malignant lymphoma; follicular malignant lymphoma; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In some embodiments, the subject is also administered an anti-cancer compound. Exemplary anti-cancer compounds include, but are not limited to, Alemtuzumab (Campath®), Alitretinoin (Panretin®), Anastrozole (Arimidex®), Bevacizumab (Avastin®), Bexarotene (Targretin®), Bortezomib (Velcade®), Bosutinib (Bosulif®), Brentuximab vedotin (Adcetris®), Cabozantinib (Cometriq™), Carfilzomib (Kyprolis™), Cetuximab (Erbitux®), Crizotinib (Xalkori®), Dasatinib (Sprycel®), Denileukin diftitox (Ontak®), Erlotinib hydrochloride (Tarceva®), Everolimus (Afinitor®), Exemestane (Aromasin®), Fulvestrant (Faslodex®), Gefitinib (Iressa®), Ibritumomab tiuxetan (Zevalin®), Imatinib mesylate (Gleevec®), Ipilimumab (Yervoy™), Lapatinib ditosylate (Tykerb®), Letrozole (Femara®), Nilotinib (Tasigna®), Ofatumumab (Arzerra®), Panitumumab (Vectibix®), Pazopanib hydrochloride (Votrient®), Pertuzumab (Perjeta™), Pralatrexate (Folotyn®), Regorafenib (Stivarga®), Rituximab (Rituxan®), Romidepsin (Istodax®), Sorafenib tosylate (Nexavar®), Sunitinib malate (Sutent®), Tamoxifen, Temsirolimus (Torisel®), Toremifene (Fareston®), Tositumomab and 131I-tositumomab (Bexxar®), Trastuzumab (Herceptin®), Tretinoin (Vesanoid®), Vandetanib (Caprelsa®), Vemurafenib (Zelboraf®), Vorinostat (Zolinza®), and Ziv-aflibercept (Zaltrap®).

In some embodiments, the subject is also administered a chemotherapeutic agent. Examples of such chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegall; dynemicin, including dynemicin A; bisphosphonates, such as clodronate; an esperamicin; as well as neocarzinostatin chromophore and related chromoprotein enediyne antibiotic chromophores, aclacinomy sins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; and pharmaceutically acceptable salts, acids or derivatives of any of the above.

In some embodiments, the subject is also administered an immunotherapeutic agent. Immunotherapy refers to a treatment that uses a subject's immune system to treat cancer, e.g. cancer vaccines, cytokines, use of cancer-specific antibodies, T cell therapy, and dendritic cell therapy.

In some embodiments, the subject is also administered an immune modulatory protein. Examples of immune modulatory proteins include, but are not limited to, B lymphocyte chemoattractant (“BLC”), C—C motif chemokine 11 (“Eotaxin-1”), Eosinophil chemotactic protein 2 (“Eotaxin-2”), Granulocyte colony-stimulating factor (“G-CSF”), Granulocyte macrophage colony-stimulating factor (“GM-CSF”), 1-309, Intercellular Adhesion Molecule 1 (“ICAM-1”), Interferon gamma (“IFN-gamma”), Interlukin-1 alpha (“IL-1 alpha”), Interleukin-1 beta (“IL-1 beta”), Interleukin 1 receptor antagonist (“IL-1 ra”), Interleukin-2 (“IL-2”), Interleukin-4 (“IL-4”), Interleukin-5 (“IL-5”), Interleukin-6 (“IL-6”), Interleukin-6 soluble receptor (“IL-6 sR”), Interleukin-7 (“IL-7”), Interleukin-8 (“IL-8”), Interleukin-10 (“IL-10”), Interleukin-11 (“IL-11”), Subunit beta of Interleukin-12 (“IL-12 p40” or “IL-12 p70”), Interleukin-13 (“IL-13”), Interleukin-15 (“IL-15”), Interleukin-16 (“IL-16”), Interleukin-17 (“IL-17”), Chemokine (C—C motif) Ligand 2 (“MCP-1”), Macrophage colony-stimulating factor (“M-CSF”), Monokine induced by gamma interferon (“MIG”), Chemokine (C—C motif) ligand 2 (“MIP-1 alpha”), Chemokine (C—C motif) ligand 4 (“MIP-1 beta”), Macrophase inflammatory protein-1-delta (“MIP-1 delta”), Platelet-derived growth factor subunit B (“PDGF-BB”), Chemokine (C—C motif) ligand 5, Regulated on Activation, Normal T cell Expressed and Secreted (“RANTES”), TIMP metallopeptidase inhibitor 1 (“TIMP-1”), TIMP metallopeptidase inhibitor 2 (“TIMP-2”), Tumor necrosis factor, lymphotoxin-alpha (“TNF alpha”), Tumor necrosis factor, lymphotoxin-beta (“TNF beta”), Soluble TNF receptor type 1 (“sTNFRI”), sTNFRIIAR, Brain-derived neurotrophic factor (“BDNF”), Basic fibroblast growth factor (“bFGF”), Bone morphogenetic protein 4 (“BMP-4”), Bone morphogenetic protein 5 (“BMP-5”), Bone morphogenetic protein 7 (“BMP-7”), Nerve growth factor (“b-NGF”), Epidermal growth factor (“EGF”), Epidermal growth factor receptor (“EGFR”), Endocrine-gland-derived vascular endothelial growth factor (“EG-VEGF”), Fibroblast growth factor 4 (“FGF-4”), Keratinocyte growth factor (“FGF-7”), Growth differentiation factor 15 (“GDF-15”), Glial cell-derived neurotrophic factor (“GDNF”), Growth Hormone, Heparin-binding EGF-like growth factor (“HB-EGF”), Hepatocyte growth factor (“HGF”), Insulin-like growth factor binding protein 1 (“IGFBP-1”), Insulin-like growth factor binding protein 2 (“IGFBP-2”), Insulin-like growth factor binding protein 3 (“IGFBP-3”), Insulin-like growth factor binding protein 4 (“IGFBP-4”), Insulin-like growth factor binding protein 6 (“IGFBP-6”), Insulin-like growth factor 1 (“IGF-1”), Insulin, Macrophage colony-stimulating factor (“M-CSF R”), Nerve growth factor receptor (“NGF R”), Neurotrophin-3 (“NT-3”), Neurotrophin-4 (“NT-4”), Osteoclastogenesis inhibitory factor (“Osteoprotegerin”), Platelet-derived growth factor receptors (“PDGF-AA”), Phosphatidylinositol-glycan biosynthesis (“PIGF”), Skp, Cullin, F-box containing complex (“SCF”), Stem cell factor receptor (“SCF R”), Transforming growth factor alpha (“TGFalpha”), Transforming growth factor beta-1 (“TGF beta 1”), Transforming growth factor beta-3 (“TGF beta 3”), Vascular endothelial growth factor (“VEGF”), Vascular endothelial growth factor receptor 2 (“VEGFR2”), Vascular endothelial growth factor receptor 3 (“VEGFR3”), VEGF-D 6Ckine, Tyrosine-protein kinase receptor UFO (“Axl”), Betacellulin (“BTC”), Mucosae-associated epithelial chemokine (“CCL28”), Chemokine (C—C motif) ligand 27 (“CTACK”), Chemokine (C—X—C motif) ligand 16 (“CXCL16”), C—X—C motif chemokine 5 (“ENA-78”), Chemokine (C—C motif) ligand 26 (“Eotaxin-3”), Granulocyte chemotactic protein 2 (“GCP-2”), GRO, Chemokine (C—C motif) ligand 14 (“HCC-1”), Chemokine (C—C motif) ligand 16 (“HCC-4”), Interleukin-9 (“IL-9”), Interleukin-17 F (“IL-17F”), Interleukin-18-binding protein (“IL-18 BPa”), Interleukin-28 A (“IL-28A”), Interleukin 29 (“IL-29”), Interleukin 31 (“IL-31”), C—X—C motif chemokine 10 (“IP-10”), Chemokine receptor CXCR3 (“I-TAC”), Leukemia inhibitory factor (“LIF”), Light, Chemokine (C motif) ligand (“Lymphotactin”), Monocyte chemoattractant protein 2 (“MCP-2”), Monocyte chemoattractant protein 3 (“MCP-3”), Monocyte chemoattractant protein 4 (“MCP-4”), Macrophage-derived chemokine (“MDC”), Macrophage migration inhibitory factor (“MIF”), Chemokine (C—C motif) ligand 20 (“MIP-3 alpha”), C—C motif chemokine 19 (“MIP-3 beta”), Chemokine (C—C motif) ligand 23 (“MPIF-1”), Macrophage stimulating protein alpha chain (“MSPalpha”), Nucleosome assembly protein 1-like 4 (“NAP-2”), Secreted phosphoprotein 1 (“Osteopontin”), Pulmonary and activation-regulated cytokine (“PARC”), Platelet factor 4 (“PF4”), Stroma cell-derived factor-1 alpha (“SDF-1 alpha”), Chemokine (C—C motif) ligand 17 (“TARC”), Thymus-expressed chemokine (“TECK”), Thymic stromal lymphopoietin (“TSLP 4-IBB”), CD 166 antigen (“ALCAM”), Cluster of Differentiation 80 (“B7-1”), Tumor necrosis factor receptor superfamily member 17 (“BCMA”), Cluster of Differentiation 14 (“CD14”), Cluster of Differentiation 30 (“CD30”), Cluster of Differentiation 40 (“CD40 Ligand”), Carcinoembryonic antigen-related cell adhesion molecule 1 (biliary glycoprotein) (“CEACAM-1”), Death Receptor 6 (“DR6”), Deoxythymidine kinase (“Dtk”), Type 1 membrane glycoprotein (“Endoglin”), Receptor tyrosine-protein kinase erbB-3 (“ErbB3”), Endothelial-leukocyte adhesion molecule 1 (“E-Selectin”), Apoptosis antigen 1 (“Fas”), Fms-like tyrosine kinase 3 (“Flt-3L”), Tumor necrosis factor receptor superfamily member 1 (“GITR”), Tumor necrosis factor receptor superfamily member 14 (“HVEM”), Intercellular adhesion molecule 3 (“ICAM-3”), IL-1 R4, IL-1 RI, IL-10 Rbeta, IL-17R, IL-2Rgamma, IL-21R, Lysosome membrane protein 2 (“LIMPII”), Neutrophil gelatinase-associated lipocalin (“Lipocalin-2”), CD62L (“L-Selectin”), Lymphatic endothelium (“LYVE-1”), MHC class I polypeptide-related sequence A (“MICA”), MHC class I polypeptide-related sequence B (“MICB”), NRG1-betal, Beta-type platelet-derived growth factor receptor (“PDGF Rbeta”), Platelet endothelial cell adhesion molecule (“PECAM-1”), RAGE, Hepatitis A virus cellular receptor 1 (“TIM-1”), Tumor necrosis factor receptor superfamily member IOC (“TRAIL R3”), Trappin protein transglutaminase binding domain (“Trappin-2”), Urokinase receptor (“uPAR”), Vascular cell adhesion protein 1 (“VCAM-1”), XEDAR, Activin A, Agouti-related protein (“AgRP”), Ribonuclease 5 (“Angiogenin”), Angiopoietin 1, Angiostatin, Cathepsin S, CD40, Cryptic family protein IB (“Cripto-1”), DAN, Dickkopf-related protein 1 (“DKK-1”), E-Cadherin, Epithelial cell adhesion molecule (“EpCAM”), Fas Ligand (FasL or CD95L), Fcg RIIB/C, FoUistatin, Galectin-7, Intercellular adhesion molecule 2 (“ICAM-2”), IL-13 R1, IL-13R2, IL-17B, IL-2 Ra, IL-2 Rb, IL-23, LAP, Neuronal cell adhesion molecule (“NrCAM”), Plasminogen activator inhibitor-1 (“PAI-1”), Platelet derived growth factor receptors (“PDGF-AB”), Resistin, stromal cell-derived factor 1 (“SDF-1 beta”), sgp130, Secreted frizzled-related protein 2 (“ShhN”), Sialic acid-binding immunoglobulin-type lectins (“Siglec-5”), ST2, Transforming growth factor-beta 2 (“TGF beta 2”), Tie-2, Thrombopoietin (“TPO”), Tumor necrosis factor receptor superfamily member 10D (“TRAIL R4”), Triggering receptor expressed on myeloid cells 1 (“TREM-1”), Vascular endothelial growth factor C (“VEGF-C”), VEGFR1, Adiponectin, Adipsin (“AND”), Alpha-fetoprotein (“AFP”), Angiopoietin-like 4 (“ANGPTL4”), Beta-2-microglobulin (“B2M”), Basal cell adhesion molecule (“BCAM”), Carbohydrate antigen 125 (“CA125”), Cancer Antigen 15-3 (“CA15-3”), Carcinoembryonic antigen (“CEA”), cAMP receptor protein (“CRP”), Human Epidermal Growth Factor Receptor 2 (“ErbB2”), Follistatin, Follicle-stimulating hormone (“FSH”), Chemokine (C—X—C motif) ligand 1 (“GRO alpha”), human chorionic gonadotropin (“beta HCG”), Insulin-like growth factor 1 receptor (“IGF-1 sR”), IL-1 sRII, IL-3, IL-18 Rb, IL-21, Leptin, Matrix metalloproteinase-1 (“MMP-1”), Matrix metalloproteinase-2 (“MMP-2”), Matrix metalloproteinase-3 (“MMP-3”), Matrix metalloproteinase-8 (“MMP-8”), Matrix metalloproteinase-9 (“MMP-9”), Matrix metalloproteinase-10 (“MMP-10”), Matrix metalloproteinase-13 (“MMP-13”), Neural Cell Adhesion Molecule (“NCAM-1”), Entactin (“Nidogen-1”), Neuron specific enolase (“NSE”), Oncostatin M (“OSM”), Procalcitonin, Prolactin, Prostate specific antigen (“PSA”), Sialic acid-binding Ig-like lectin 9 (“Siglec-9”), ADAM 17 endopeptidase (“TACE”), Thyroglobulin, Metalloproteinase inhibitor 4 (“TIMP-4”), TSH2B4, Disintegrin and metalloproteinase domain-containing protein 9 (“ADAM-9”), Angiopoietin 2, Tumor necrosis factor ligand superfamily member 13/Acidic leucine-rich nuclear phosphoprotein 32 family member B (“APRIL”), Bone morphogenetic protein 2 (“BMP-2”), Bone morphogenetic protein 9 (“BMP-9”), Complement component 5a (“C5a”), Cathepsin L, CD200, CD97, Chemerin, Tumor necrosis factor receptor superfamily member 6B (“DcR3”), Fatty acid-binding protein 2 (“FABP2”), Fibroblast activation protein, alpha (“FAP”), Fibroblast growth factor 19 (“FGF-19”), Galectin-3, Hepatocyte growth factor receptor (“HGF R”), IFN-alpha/beta R2, Insulin-like growth factor 2 (“IGF-2”), Insulin-like growth factor 2 receptor (“IGF-2 R”), Interleukin-1 receptor 6 (“IL-1R6”), Interleukin 24 (“IL-24”), Interleukin 33 (“IL-33”, Kallikrein 14, Asparaginyl endopeptidase (“Legumain”), Oxidized low-density lipoprotein receptor 1 (“LOX-1”), Mannose-binding lectin (“MBL”), Neprilysin (“NEP”), Notch homolog 1, translocation-associated (Drosophila) (“Notch-1”), Nephroblastoma overexpressed (“NOV”), Osteoactivin, Programmed cell death protein 1 (“PD-1”), N-acetylmuramoyl-L-alanine amidase (“PGRP-5”), Serpin A4, Secreted frizzled related protein 3 (“sFRP-3”), Thrombomodulin, Toll-like receptor 2 (“TLR2”), Tumor necrosis factor receptor superfamily member 10A (“TRAIL R1”), Transferrin (“TRF”), WIF-1ACE-2, Albumin, AMICA, Angiopoietin 4, B-cell activating factor (“BAFF”), Carbohydrate antigen 19-9 (“CA19-9”), CD 163, Clusterin, CRT AM, Chemokine (C—X—C motif) ligand 14 (“CXCL14”), Cystatin C, Decorin (“DCN”), Dickkopf-related protein 3 (“Dkk-3”), Delta-like protein 1 (“DLL1”), Fetuin A, Heparin-binding growth factor 1 (“aFGF”), Folate receptor alpha (“FOLR1”), Furin, GPCR-associated sorting protein 1 (“GASP-1”), GPCR-associated sorting protein 2 (“GASP-2”), Granulocyte colony-stimulating factor receptor (“GCSF R”), Serine protease hepsin (“HAI-2”), Interleukin-17B Receptor (“IL-17B R”), Interleukin 27 (“IL-27”), Lymphocyte-activation gene 3 (“LAG-3”), Apolipoprotein A-V (“LDL R”), Pepsinogen I, Retinol binding protein 4 (“RBP4”), SOST, Heparan sulfate proteoglycan (“Syndecan-1”), Tumor necrosis factor receptor superfamily member 13B (“TACI”), Tissue factor pathway inhibitor (“TFPI”), TSP-1, Tumor necrosis factor receptor superfamily, member 10b (“TRAIL R2”), TRANCE, Troponin I, Urokinase Plasminogen Activator (“uPA”), Cadherin 5, type 2 or VE-cadherin (vascular endothelial) also known as CD144 (“VE-Cadherin”), WNT1-inducible-signaling pathway protein 1 (“WISP-1”), and Receptor Activator of Nuclear Factor κ B (“RANK”).

In some embodiments, the subject is also administered an immune checkpoint inhibitor. Immune Checkpoint inhibition broadly refers to inhibiting the checkpoints that cancer cells can produce to prevent or downregulate an immune response. Examples of immune checkpoint proteins include, but are not limited to, CTLA4, PD-1, PD-L1, PD-L2, A2AR, B7-H3, B7-H4, BTLA, KIR, LAG3, TIM-3 or VISTA. Immune checkpoint inhibitors can be antibodies or antigen binding fragments thereof that bind to and inhibit an immune checkpoint protein. Examples of immune checkpoint inhibitors include, but are not limited to, nivolumab, pembrolizumab, pidilizumab, AMP-224, AMP-514, STI-A1110, TSR-042, RG-7446, BMS-936559, MEDI-4736, MSB-0020718C, AUR-012 and STI-A1010.

In some embodiments, a composition provided herein (e.g., a vaccine composition provided herein) is administered prophylactically to prevent cancer and/or a CMV infection. In some embodiments, the vaccine is administered to inhibit tumor cell expansion. The vaccine may be administered prior to or after the detection of cancer cells or CMV infected cells in a patient. Inhibition of tumor cell expansion is understood to refer to preventing, stopping, slowing the growth, or killing of tumor cells. In some embodiments, after administration of a vaccine comprising peptides, nucleic acids, antibodies or APCs described herein, a proinflammatory response is induced. The proinflammatory immune response comprises production of proinflammatory cytokines and/or chemokines, for example, interferon gamma (IFN-γ) and/or interleukin 2 (IL-2). Proinflammatory cytokines and chemokines are well known in the art.

Conjunctive therapy includes sequential, simultaneous and separate, and/or co-administration of the active compounds in such a way that the therapeutic effects of the first agent administered have not entirely disappeared when the subsequent treatment is administered. In some embodiments, the second agent may be co-formulated with the first agent or be formulated in a separate pharmaceutical composition.

Actual dosage levels of the active ingredients in the pharmaceutical compositions provided herein may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

In some aspects, provided herein is a method of identifying a subject suitable for a therapy provided herein (methods of treating a CMV infection and/or a cancer in a subject comprising administering to the subject a pharmaceutical composition provided herein). In some embodiments, the method comprises isolating a sample from the subject (e.g., a blood sample, a tissue sample, a tumor sample) and detecting the presence of a CMV epitope listed in Table 1 in the sample. In some embodiments the epitope is detected using an ELISA assay, a western blot assay, a FACS assay, a fluorescent microscopy assay, an Edman degradation assay and/or a mass spectrometry assay (e.g., protein sequencing). In some embodiments, the presence of the CMV epitope is detected by detecting a nucleic acid encoding the CMV epitope. In some embodiments, the nucleic acid encoding the CMV epitope is detected using a nucleic acid probe, a nucleic acid amplification assay and/or a sequencing assay.

Examples of nucleic acid amplification assays that can be used in the methods provided herein include, but are not limited to polymerase chain reaction (PCR), LATE-PCR, ligase chain reaction (LCR), strand displacement amplification (SDA), transcription mediated amplification (TMA), self-sustained sequence replication (3SR), Qβ replicase based amplification, nucleic acid sequence-based amplification (NASBA), repair chain reaction (RCR), boomerang DNA amplification (BDA) and/or rolling circle amplification (RCA).

In some embodiments the product of the amplification reaction is detected as an indication of the presence and/or identity of the bacteria in the sample. In some embodiments, the amplification product is detected after completion of the amplification reaction (i.e., endpoint detection). Examples of end-point detection methods include gel-electrophoresis based methods, probe-binding based methods (e.g., molecular beacons, HPA probes, lights-on/lights-off probes) and double-stranded DNA binding fluorescent-dye based methods (e.g., ethidium bromide, SYBR-green). In some embodiments, the amplification product is detected as it is produced in the amplification reaction (i.e., real-time detection). Examples of real-time detection methods include probe-binding based methods (e.g., molecular beacons, TaqMan probes, scorpion probes, lights-on/lights-off probes) and double-stranded DNA binding fluorescent-dye based methods (e.g., ethidium bromide, SYBR-green). In some embodiments, the product of the amplification reaction is detected and/or identified by sequencing (e.g., through the use of a sequencing assay described herein).

In some embodiments, the detection of the nucleic acid sequence comprises contacting the nucleic acid sequence with a nucleic acid probe that hybridizes specifically to the nucleic acid sequence. In some embodiments, the probe is detectably labeled. In some embodiments, the probe is labeled (directly or indirectly) with a fluorescent moiety. Examples of fluorescent moieties useful in the methods provided herein include, but are not limited to Allophycocyanin, Fluorescein, Phycoerythrin, Peridinin-chlorophyll protein complex, Alexa Fluor 350, Alexa Fluor 405, Alexa Fluor 430, Alexa Fluor 488, Alexa Fluor 514, Alexa Fluor 532, Alexa Fluor 546, Alexa Fluor 555, Alexa Fluor 568, Alexa Fluor 594, Alexa Fluor 633, Alexa Fluor 635, Alexa Fluor 647, Alexa Fluor 660, Alexa Fluor 680, Alexa Fluor 700, Alexa Fluor 750, Alexa Fluor 790, GFP, RFP, YFP, EGFP, mPlum, mCherry, mOrange, mKO, EYFP, mCitrine, Venus, YPet, Emerald, Cerulean and CyPet. In some embodiments, the probe is a molecular beacon probe, a molecular torch probe, a TaqMan probes, a SDA probe, a scorpion probe, a HPA probe, or a lights on/lights off probe.

In some embodiments, the nucleic acid sequence is detected by sequencing (e.g., whole genome sequencing, transcriptome sequence and/or targeted gene sequencing). Examples of sequencing processes that can be used in the methods provided herein include, but are not limited to, chain termination sequencing, massively parallel signature sequencing, ion semiconductor sequencing, polony sequencing, illumina sequencing, sequencing by ligation, sequencing by synthesis, pyrosequencing, single-molecule real-time sequencing, SOLiD sequencing, DNA nanoball sequencing, heliscope single molecule sequencing, single molecule real time sequencing, 454 sequencing, nanopore sequencing, tunneling currents DNA sequencing or sequencing by hybridization.

In some embodiments, the methods provided herein further comprise treating the identified subject using a therapeutic method provided herein (e.g., by administering to the subject a pharmaceutical composition provided herein).

### EXAMPLES

### Example 1: Dynamics of the Emergence of Genetic Variants of CMV Following Viral Reactivation in HSCT Recipients

Twenty six patients undergoing allogeneic hematopoietic stem cell transplantation (HSCT) were enrolled for this study. The clinical characteristics of these patients are listed in Table 4. All patients received a T cell-replete bone marrow or G-CSF-mobilized peripheral blood stem cell graft and none had in vivo T cell depletion. CMV-seropositive patients or patients who received a transplant from a seropositive donor were treated prophylactically with high dose acyclovir from day −5 to day 28 or until discharge, then with valacicolvir until day 100. Patients with CMV DNAemia in plasma of >600 copies/mL were treated with ganciclovir twice daily for 14 days, followed by once daily maintenance until plasma DNAemia was <600 copies/mL; or with valganciclovir at 900 mg twice daily followed by 900 mg once daily for maintenance. Foscarnet was used to treat patients who were nonresponsive or displayed significant toxicity from ganciclovir. Of the 26 HSCT recipients enrolled for this study, 17 displayed evidence of viral reactivation, as defined by CMV DNAemia >600 copies/ml. Early CMV reactivation developed in 16 of these patients, while late CMV was detected in four. Two of these patients developed CMV-associated disease: one colitis and one enteritis. Fourteen of the seventeen displayed an unstable CMV-specific immune response (as assessed by CMV-QuantiFERON assay). Nine patients included in the current study demonstrated CMV-immune reconstitution without evidence of viral reactivation.

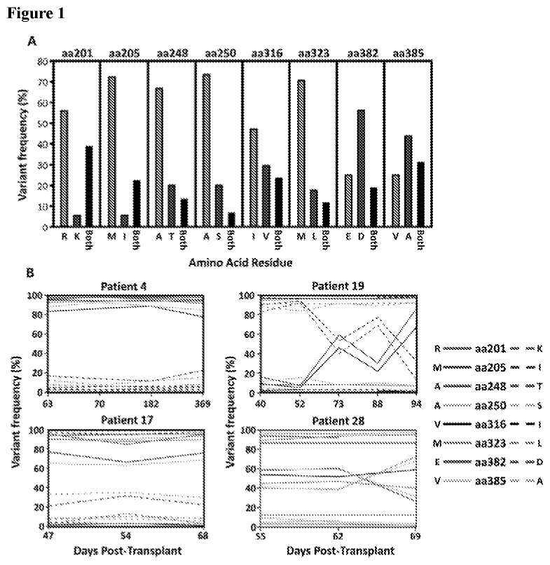

To delineate the impact of the emergence of genetic variants on T cell immune reconstitution in this cohort of HSCT recipients, eight different HLA class I restricted CD8+ T cell epitopes were chosen from the Immediate Early (IE-1) protein of CMV. Using the Genbank database, a series of variant sequences were identified for each of these epitopes. A pyrosequencing analysis was designed to identify the single nucleotide polymorphisms (SNPs) within the CMV-encoded CD8+ T cell epitopes. Initially, these SNP analyses were carried out at the peak of viral load in all HSCT recipients who showed CMV reactivation. The amino acid residue at each variant position was extrapolated based upon the nucleotide sequence. Data in FIG. 1A represents the proportion of recipients showing either one or both amino acids at each position. Data was corrected for error rates at each position as outlined in the Materials and Methods. Bias was observed in amino acid usage at certain positions, particularly the preferentially usage of R, M, A, A and M residues at positions 201, 205, 248, 250 and 323, respectively, significantly more variation was noted at other residues. This analysis also revealed a high proportion of HSCT recipients had multiple IE-1 variants following reactivation, whereby 6-35% of the samples at each position were associated with the detection of both amino acids and 9-of-17 HSCT recipients showed definitive evidence of mixed infection characterized by the concurrent detection of both variant residues on at least one position. The stability of the viral variants was assessed over time, using longitudinal plasma samples during viral reactivation from 15 of the 17 HSCT recipients. Representative longitudinal analysis of all SNPs assessed from 4 recipients is shown in FIG. 1B. Whilst some HSCT recipients showed very little change in the pattern of SNP expression either following detection of predominantly single variant (recipient 4) or likely co-infection (recipient 17), other HSCT recipients demonstrate changes in SNP frequency during periods of viral reactivation (recipients 19 and 28); suggesting the potential impact of immunological selective pressure on the dominant viral isolates in the peripheral blood of these HSCT recipients.

### Example 2: Impact of Co-Infection on the T Cell Kinetics

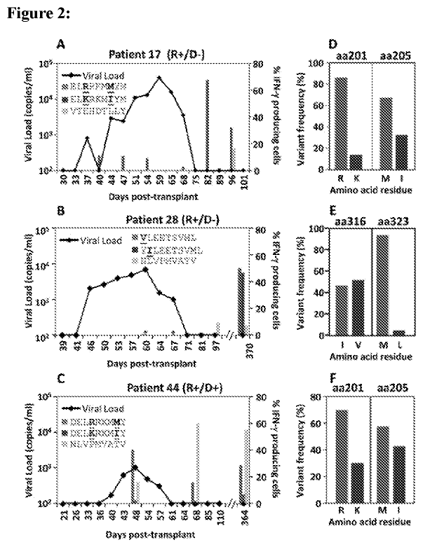

To assess the impact of epitope variation and co-infection on IE-1 specific T cell immunity, PBMC samples from HSCT recipients showing evidence of viral reactivation were stimulated with all potentially HLA-matched variant peptide epitopes then cultured in vitro for two weeks in the presence of IL-2. PBMC from nine HSCT recipients showing immune reconstitution with no evidence of CMV reactivation were also stimulated with HLA-matched variant peptide epitopes (Table 2). As a control, PBMC were stimulated with at least two conserved HLA matched epitopes. Representative longitudinal analysis from three of these patients overlaid with viral reactivation kinetics is shown in FIG. 2A-C. An overall summary of the number of HSCT recipients tested for each epitope and the number of responding HSCT recipients is shown in Table 3. Interestingly, these observations suggested that while some patients could efficiently recognize multiple viral variants detected by pyrosequencing analysis (represented by patient 28, FIGS. 2(B and E) others showed preferential recognition, in some instances targeted against subdominant epitope variants. As evident in FIG. 2D, pyrosequencing analysis revealed that the IE-1 sequence in recipient 17 at amino acid residues 201 and 205 was dominated by the amino acid residues R and M, which would correspond to the ELRRKMMYM epitope in HLA-B8 individuals. Despite this, recipient 17 only generated a T cell response against the subdominant ELKRKMIYM variant (FIG. 2A).

TABLE 2
List of Exemplary IE-1 Epitope Variants

| | | | | Amino |
|---|---|---|---|---|
| | | | | Acid |
| | HLA | | | Variant; |
| | Restric- | Sequence | Major Epitope | Position |
| Epitope | tion | Position | Varient | [symbol] |
| KARAKKDELR | A31 | 192-201 | KARAKKDELK | R/K P10 |
| ARAKKDELR | B27 | 193-201 | ARAKKDELK | R/K P9 |
| RRKMMYMYCR | B27 | 201-210 | KRKMIYMYCR | R/K |
| | | | | P1 |
| | | | | M/I |
| DELRRKMMY | B18; B44 | 198-206 | DELKRKMIY | R/K |
| | | | | P4; |
| | | | | [symbol] |
| ELRRKMMYM | B8 | 199-207 | ELKRKMIYM | R/K |
| | | | | P3; |
| | | | | [symbol] |
| AYAQKIFKIL | A23 | 248-257 | TYSQKIFK1L | A/T |
| | | | | P1; |
| | | | | A/S |
| VLEETSVML | A2 | 316-324 | YILEETSVML | V/I |
| | | | | P1 or |
| | | | | P2; |
| EEAIVAYTL | B18; B44 | 381-390 | EDAIAAYTL | E/D |
| | | | | P2; |
| | | | | [symbol] |

[symbol]

indicates data missing or illegible when filed

Interestingly, recipient 17 also showed the absence of a detectable response against the immunodominant conserved T cell epitope, VTEHDTTLY during viral reactivation and failed to generate a T cell response against the dominant ELRRKMMYM variant even after resolution of viral infection. Similar observations were evident for recipient 44 (FIG. 2F). It was possible to detect sequences encoding both of the HLA-B44 variants, but a response against the DELKRKMIY variant during viral reactivation was not detected. Interestingly, these observations were also evident in other HLA-B44-positive HSCT recipients for both of the HLA-B44 restricted epitopes (Table 3). This was particularly evident for the EDAIAAYTL variant that could be detected in 6 of 7 HLA B44-positive HSCT recipients but failed to induce a significant T cell response in any recipient.

TABLE 3
Summary of CMV-specific peptide epitope recognition by HSCT recipients

| | Reactivation | | | | |
|---|---|---|---|---|---|
| | | Number of | | | |
| | | HLA | | | |
| | | Matched | | No Reactivation | |
| | Number of | Recipients | | Number of | |
| | HLA | with | | HLA | |
| Peptide | Matched | sequence | Number of | Matched | Number of |
| Sequence | Recipients | detected | Responders# | Recipients | Responders# |
| VLEETSVML | 12 | 7 | 3 | 5 | 1 |
| YILEETSVML | 12 | 5 | 4 | 5 | 2 |
| DELDRKMMY | 7 | 5 | 2 | 4 | 0 |
| DELKRKMIY | 7 | 3 | 1 | 4 | 0 |
| EEAIAVAYL | 7 | 4 | 2 | 4 | 0 |
| EDAIAAYTL | 7 | 6 | 0 | 4 | 0 |
| ELRRKMMYM | 2 | 2 | 1 | 2 | 2 |
| ELKRKMIYM | 2 | 1 | 2 | 2 | 2 |
| AYAQKIFKIL | 1 | 0 | 1 | 1 | 0 |
| TYSQKIFKIL | 1 | 1 | 1 | 1 | 1 |
| KARAKKDELR | 1 | 1 | 0 | 1 | 0 |
| KARAKKDELK | 1 | 0 | 0 | 1 | 0 |
| ARAKKDELK | 1 | 1 | 1 | 1 | 0 |
| ARAKKDELR | 1 | 1 | 1 | 1 | 0 |
| KRKMIYMCYR | 1 | 0 | 0 | 1 | 1 |
| RRKMMYWMCYR | 1 | 1 | 1 | 1 | 1 |
| FMDILTTCV | 12 | N.D. | 5 | 5 | 0 |
| NLVPMVATV | 12 | N.D. | 8 | 5 | 3 |
| RPHERNGFTVL | 1 | N.D. | 1 | 1 | 1 |
| TPRVTGGGAM | 1 | N.D. | 1 | 1 | 1 |
| VTEHDTLLY | 3 | N.D. | 3 | 4 | 3 |
| QIKVRVDMV | 2 | N.D. | 1 | 1 | 1 |
| YSEHPTFTSQY | 0 | N.D. | 0 | 2 | 2 |

N.D. Not Done
#Patients with >5% of CD8+ T cells producing IFN-γ following recall after two weeks of culture were considered Responders

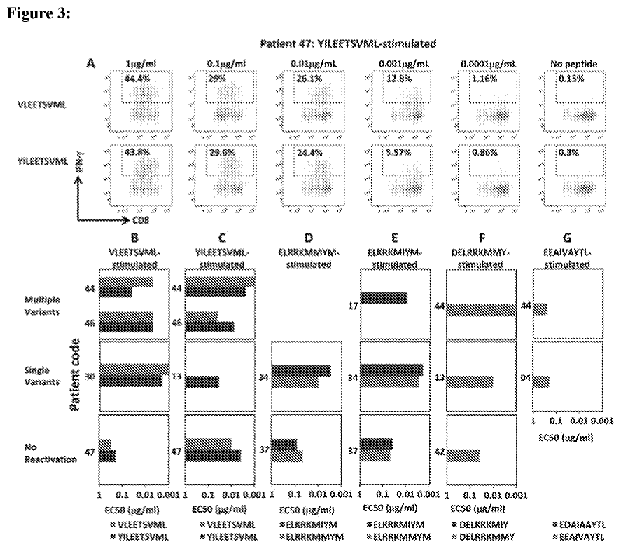

To further assess the recognition of epitope variants in our recipient cohort, cultured T cells from all HSCT recipients were stimulated with serial dilutions of both the cognate and variant peptide and assessed for the production of IFN-γ. The effective concentration (EC) 50 was then calculated based upon the concentration of peptide required to induce 50% of maximal IFN-γ production. Representative analysis following recall of a YILEETSVML-stimulated T cell culture with 10-fold serial dilutions of the VLEETSVML and YILEETSVML epitope variants is shown in FIG. 3A. While T cells specific for HLA-A2 restricted epitopes (VLEETSVML and YILEETSVML) consistently recognized both variants with similar efficiency (FIGS. 3B and C), cross-reactivity towards the HLA-B8 epitopes, ELRRKMMYM and ELKRKMIYM, was patient-dependent, characterized by preference for a single variant in some individuals (recipient 17) and cross-reactive in others (recipients 34 and 37) (FIGS. 3D and E). There was no evidence of cross-reactivity in T cells specific for the two B44 restricted epitopes, DELRRKMMY and EEAIVAYTL which displayed preferential bias for a single variant, irrespective of evidence for exposure to multiple variants (FIGS. 3F and G). These observations further demonstrate that exposure to multiple viral isolates does not automatically lead to the efficient induction of cross-reactive T cell immunity and repertoire “holes” may exist across genetically unrelated individuals.

### Example 3: The Impact of Exposure to Multiple Viral Isolates on Viral Control

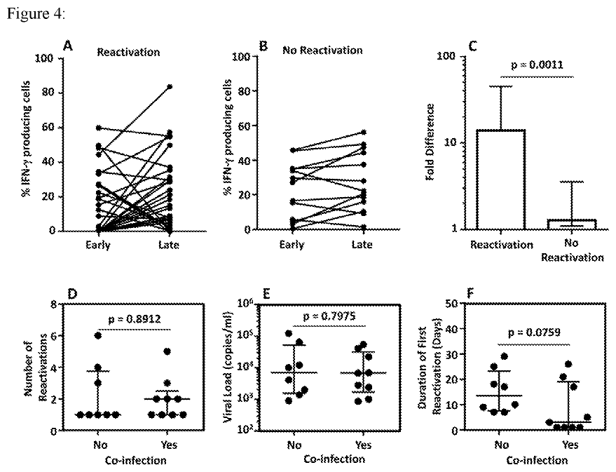

To determine if the reconstitution of the CMV-specific T cell response directed towards both variant IE-1 and/or conserved epitopes was associated with viral reactivation, the frequency of CD8+ T cells specific for both IE-1 variant epitopes and conserved epitopes early (90-106 days) and late (>180 days) post-transplant in HSCT recipients with and without evidence of reactivation was compared. Pairwise analysis of the frequency of all detectable CMV-specific T cell responses early and late post-transplant demonstrated that HSCT recipients with evidence of viral reactivation (FIG. 4A) showed less stability in their T cell responses compared to HSCT recipients without reactivation (FIG. 4B). Additionally, HSCT recipients with reactivation showed significantly greater fold differences in the frequency of CMV-specific T cells between early and late responses compared to HSCT recipients with no reactivation, who displayed very little change in the frequency of their virus-specific T cell responses (FIG. 4C). To further assess the impact of reactivation with multiple viral isolates on viral control (i) the number of viral reactivations; (ii) the peak viral load and (iii) duration of the first viral reactivations in HSCT recipients with evidence of single or multiple variants in their peripheral blood was compared. These analyses revealed no significant differences in the number of viral reactivations (FIG. 4D), in the peak viral load (FIG. 4E) or in the duration of reactivation (FIG. 4F) from patients with and without evidence of multiple viral isolates. These observations suggest that whilst the induction of variant specific immunity may play a role in the control of viral reactivation following reactivation with multiple isolates of CMV, the capacity to induce stable CMV-specific immune reconstitution to either conserved epitopes or via cross-reactive responses was more relevant for the efficient control of CMV reactivation following HSCT.

TABLE 4
Clinical Characteristics of HSCT Recipients included in this study

| | | | | | | |
|---|---|---|---|---|---|---|
| | | | | | CMV load > 600 | |
| | Recipient/ | | Episodes of | Maximal | copies/mL | |
| | Donor | | CMV | CMV | (days post- | CMV |
| Code | Serostatus | HLA Type | Reactivation | titre | transplant) | Disease |

Patients with CMV reactivation*

| | | | | | | |
|---|---|---|---|---|---|---|
| 04 | R+/D− | A2 A29 B44 B51 Cw1 | 4 | 10000 | 60-70; 144-158; | Yes: CMV |
| | | | | | 189-195; 363-391 | colitis |
| 06 | R+/D− | A23 A26 B39 B51 Cw2 | 1 | 900 | 64-71 | No |
| 13 | R+/D− | A2 A29 B44 B62 Cw3 | 2 | 12000 | 33-67; 77-84 | No |
| 14 | R+/D+ | A11 A31 B7 B60 | 6 | 120000 | 46-55; 139-178; | Yes; |
| | | | | | 192-196; 213- | CMV |
| | | | | | 217; 249-269; | enteritis |
| | | | | | 286-314 | |
| 16 | R+/D− | A2 A24 B15 B27 Cw2 Cw3 | 1 | 870 | 69 | No |
| 17 | R+/D− | A1 A24 B08 B39 Cw7 | 2 | 40000 | 37; 44-68 | No |
| 19 | R+/D+ | A2 A24 B44 Cw5 | 3 | 55000 | 32-64; 73-80; 88- | No |
| | | | | | 92 | |
| 25 | R+/D− | A2 A3 B35 B62 Cw3 Cw10 | 2 | 2400 | 59; 95-102 | No |
| 26 | R+/D− | A2 A33 B14 B15 Cw3 Cw8 | 3 | 4100 | 35-60; 81-88; | No |
| | | | | | 273-277 | |
| 28 | R+/D− | A2 A24 B44 Cw5 Cw6 | 1 | 6800 | 46-67 | No |
| 30 | R+/D+ | A2 A24 B13 B60 Cw3 Cw4 | 1 | 64000 | 314-332 | No |
| 32 | R+/D− | A2 B13 B40 Cw3 Cw6 | 5 | 22000 | 39; 49-63; 151- | No |
| | | | | | 157; 179; 192-237 | |
| 34 | R+/D− | A1 A33 B8 B14 Cw7 Cw8 | 1 | 2000 | 57-64 | No |
| 38 | R+/D+ | A1 A24 B41 B57 Cw6 | 1 | 1400 | 75-92 | No |
| 39 | R+/D− | A2 A29 B44 Cw5 | 1 | 6900 | 45-62 | No |
| 44 | R+/D+ | A2 A32 B18 B44 Cw5 Cw7 | 1 | 1000 | 43-48 | No |
| 46 | R+/D− | A2 B27 B44 Cw2 Cw5 | 2 | 2800 | 32-35; 53 | No |

Patients without CMV reactivation

| | | | | | | |
|---|---|---|---|---|---|---|
| 01 | R+/D− | A1 A3 B27 B60 Cw2 Cw3 | N.A | N.A | N.A | No |
| 07 | R−/D+ | A1 A2 B08 B15 Cw3 Cw7 | N.A | N.A | N.A | No |
| 15 | R+/D− | A3 A31 B7 B60 Cw3 Cw7 | N.A | N.A | N.A | No |
| 36 | R+/D− | A1 A2 B35 B62 Cw3 Cw4 | N.A | N.A | N.A | No |
| 37 | R+/D− | A2 A23 B15 B44 Cw4 Cw7 | N.A | N.A | N.A | No |
| 42 | R+/D+ | A2 A23 B15 B44 Cw4 Cw7 | N.A | N.A | N.A | No |
| 43 | R+/D+ | A1 A26 B44 B13 Cw7 | N.A | N.A | N.A | No |
| 45 | R+/D− | A1 A2 B37 B44 Cw5 Cw6 | N.A | N.A | N.A | No |
| 47 | R+/D+ | A2 B7 B44 Cw5 Cw7 | N.A | N.A | N.A | No |

N.A. Not Applicable
*CMV reactivation defined as CMV DNAemia > 600 copies/ml

All publications, patents, patent applications and sequence accession numbers mentioned herein are hereby incorporated by reference in their entirety as if each individual publication, patent or patent application was specifically and individually indicated to be incorporated by reference. In case of conflict, the present application, including any definitions herein, will control.

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating a cancer in a subject, comprising administering to the subject a pharmaceutical composition comprising cytotoxic T cells (CTLs) comprising a T cell receptor (TCR) that specifically binds to a peptide comprising an epitope listed in Table 1 presented on a class I MHC.

2. A method of treating a cytomegalovirus (CMV) infection in a subject, comprising administering to the subject a pharmaceutical composition comprising cytotoxic T cells (CTLs) comprising a T cell receptor (TCR) that specifically binds to a CMV peptide comprising an epitope listed in Table 1 presented on a class I MHC.

3. The method of claim 1 or 2, wherein the CTLs are autologous to the subject.

4. The method of claim 1 or 2, wherein the CTLs are not autologous to the subject.

5. The method of claim 4, wherein the CTLs are obtained from a CTL library or bank.

6. A method of inducing proliferation of CMV-specific cytotoxic T cells (CTLs) comprising incubating a sample comprising CTLs and antigen-presenting cells (APCs) that present a CMV peptide comprising an epitope listed in Table 1 thereby inducing proliferation peptide-specific CTLs in the sample.

7. The method of claim 6, wherein the sample further comprises one or more cytokines.

8. The method of claim 6 or 7, wherein the APCs are B cells.

9. The method of claim 6 or 7, wherein the APCs are antigen presenting T-cells.

10. The method of claim 6 or 7, wherein the APCs are dendritic cells.

11. The method of claim 6 or 7, wherein the APCs are aK562 cells.

12. The method of any one of claims 6 to 10, wherein the sample comprises peripheral blood mononuclear cells (PBMCs).

13. The method of any one of claims 1 to 10, wherein the T-cells are cytotoxic T-cells.

14. The method of any claims 1 to 13, wherein the CMV peptide is no more than 20 amino acids in length.

15. The method of claim 14, wherein the CMV peptide is no more than 15 amino acids in length.

16. The method of claim 14, wherein the CMV peptide is no more than 10 amino acids in length.

17. The method of any one of claims 1 to 16, wherein the CMV peptide comprises a sequence of KARAKKDELR.

18. The method of any one of claims 1 to 16, wherein the CMV peptide comprises a sequence of ARAKKDELR.

19. The method of any one of claims 1 to 16, wherein the CMV peptide comprises a sequence of RRKMMYMYCR.

20. A peptide comprising an amino acid sequence listed in Table 1, wherein the peptide does not comprise more than 30 contiguous amino acids of a CMV protein.

21. The peptide of claim 20, wherein the amino acid sequence listed in Table 1 is KARAKKDELR, ARAKKDELR or RRKMMYMYCR.

22. The peptide of claim 20 or 21, wherein peptide comprises two or more sequences listed in Table 1.

23. A vaccine composition comprising a peptide of any one of claims 20 to 22.

24. The vaccine composition of claim 23, further comprising an adjuvant.

25. A method of treating and or preventing cancer in a subject, comprising administering to a subject a vaccine composition of claim 23 or 24.

26. A method of treating and or preventing a CMV infection in a subject, comprising administering to a subject a vaccine composition of claim 23 or 24.

27. An antigen-presenting cell (APC) comprising a peptide of any one of claims 20 to 22 presented on a class I MHC.

28. The APC of claim 27, wherein the APC is an antigen-presenting T-cell.

29. The APC of claim 27, wherein the APC is a dendritic cell.

30. The APC of claim 27, wherein the APC is a B cell.

31. The APC of claim 27, wherein the APC is an artificial APC.

32. The APC of claim 31, wherein the artificial APC is an aK562 cell.

33. A method of producing an antigen-presenting cells (APC) that presents a CMV peptide comprising incubating an antigen-presenting cell with the peptide of any one of claims 20 to 22 or a nucleic acid encoding a peptide of any one of claims 20 to 22.

34. The method of claim 33, wherein the APC is an antigen presenting T-cell.

35. The method of claim 33, wherein the APC is a dendritic cell.

36. The method of claim 33, wherein the APC is a B cell.

37. The method of claim 33, wherein the APC is an artificial APC.

38. The method of claim 33, wherein the artificial APC is an aK562 cell.

39. A method of treating or preventing cancer in a subject, comprising administering to the subject the APCs of any one of claims 27 to 32.

40. The method of claim 39, wherein the APC is autologous to the subject.

41. The method of claim 39, wherein the APC is not autologous to the subject.

42. A method of treating or preventing a CMV infection in a subject, comprising administering to a subject the APCs of any one of claims 37 to 41.

43. The method of claim 42, wherein the APC is autologous to the subject.

44. The method of claim 42, wherein the APC is not autologous to the subject.

45. A nucleic acid encoding the peptide of any one of claims 20 to 22.

46. The nucleic acid of claim 45, wherein the nucleic acid is an expression vector.

47. The nucleic acid of claim 46, wherein the expression vector is a viral vector.

48. The nucleic acid of claim 47, wherein the viral vector is an adenovirus-based expression vector.

49. A vaccine composition comprising a nucleic acid of any one of claims 45 to 48.

50. A method of treating and or preventing cancer in a subject, comprising administering to the subject the vaccine composition of claim 49.

51. A method of treating or preventing a CMV infection in a subject, comprising administering to the subject the vaccine composition of claim 49.

52. An antibody or antigen-binding fragment thereof that binds to a CMV epitope listed Table 1.

53. The antibody or antigen-binding fragment thereof of claim 52, wherein the antibody or antigen-binding fragment thereof is:
a full length immunoglobulin molecule;
an scFv;
a Fab fragment;
an Fab′ fragment;
an F(ab′)2;
an Fv;
a camelid antibody; or
a disulfide linked Fv.

54. A method of treating cancer in a subject, comprising administering to the subject an antibody or antigen-binding fragment thereof of claim 52 or claim 53.

55. A method of treating a CMV infection in a subject, comprising administering to the subject an antibody or antigen-binding fragment thereof of claim 52 or claim 53.

56. A T cell expressing a T cell receptor (TCR) that binds to a peptide comprising an epitope listed in Table 1 presented on a major histocompatibility complex (MHC).

57. The T cell of claim 56, wherein the T cell is a cytotoxic T cell (CTL).
